(19) United States
(12) Patent Application Publication
(10) Pub. No.: US 2023/0277765 A1
(43) Pub. Date: Sep. 7, 2023

(54) INSULIN INFUSION SET

(71) Applicant: Tandem Diabetes Care, Inc., San Diego, CA (US)

(72) Inventors: Amrith Karunaratne, Irvine, CA (US); David S. Gillett, Irvine, CA (US)

(21) Appl. No.: 18/115,316

(22) Filed: Feb. 28, 2023

Related U.S. Application Data
(60) Provisional application No. 63/314,901, filed on Feb. 28, 2022.

(51) Int. Cl. A61M 5/165

(52) U.S. Cl. CPC A61M 5/165; A61M 2005/1652

(57) ABSTRACT

Embodiments of devices and methods to maintain preservative concentration in a medication delivered using a medical device are provided. A barrier layer can be used to prevent migration of preservatives. A vent can be used to allow release of preservatives prior to delivery to the patient. An absorbent element can be used to maintain preservative concentration at a desired level. A filter can be used to capture particulates from the medication prior to delivery to a patient.

### CROSS REFERENCE TO RELATED APPLICATIONS

The present application claims the benefit of U.S. Provisional Application No. 63/314,901, filed Feb. 28, 2022, which may be related to PCT Application No. PCT/US2021/048015, filed Aug. 27, 2021. U.S. application Ser. No. 17/446,271, filed Aug. 27, 2021; U.S. Application Ser. No. 15/943,517, filed Apr. 2, 2018, now U.S. Pat. No. 10,413,658; PCT Application No. PCT/US2019/060602, filed Nov. 8, 2019; and U.S. application Ser. No. 17/289,009, filed Nov. 8, 2019, the entire disclosures of which are herein incorporated by reference in their entireties.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Infusion sets are known in the art for delivering a medicament to a patient at a subcutaneous site. Infusion Sets generally consist of three major components: a unique connection to fluidically connect to a source of medicament, normally a pump or other source of fluid under pressure; a flexible tube consisting of one or more layers of engineering polymer and of appropriate length; and an infusion Set that provides Subcutaneous access to the patient.

The most common use for infusion sets as described herein is for the delivery of insulin to a diabetic patient.

While technical improvements to infusion pumps have been significant, infusion set patency, ease of use, sterility, safety and user comfort are areas that have gone largely unaddressed, despite the growing number of complaints by users. A majority of infusion sets sold today may kink or otherwise become closed to fluid delivery (occlusion). Occlusion may occur for a number of reasons, such as insertion procedure, infusion set placement site, user activity, adhesive failure (resulting in de-lamination and shearing), etc. Unfortunately, due to the relatively slow rate of delivery of insulin by the infusion pump in most circumstances and/or the unreliability of pump overpressure alarms, a kink or closure in the cannula may not be discovered until it is too late (i.e., the patient goes into a state of hyperglycemia).

This problem is compounded by the relatively new introduction of longer-term in-dwelling catheters (infusion sets). These new infusion sets embody design features, materials and fabrication methodologies not foreseen in prior art and, in fact, not available to practitioners at the time.

Insulin delivery systems have become an important mechanism for treating diabetes. However, the protein insulin, including insulin analogs, is an inherently unstable molecule. In addition to chemical changes that can occur as the result general acid hydrolysis, disulfide scrambling, and other chemical transformations, insulin can be prone to self-associate and precipitate from solution under certain conditions.

To counteract this physical instability of insulin, many insulin formulations have been optimized to inhibit insulin precipitation during storage. For example, insulin formulations often include phenolic preservatives. Phenolic preservatives are important to maintain within a formulation because they induce an aggregation-resistant conformation (R₆) when they complex with insulin.

Preservative loss in an insulin delivery system is often a two-stage process that includes: (1) absorption of the preservative into fluid-path materials; and (2) evaporation of the fluid preservative from these materials into the air. The absorption rate is generally important in the short term, as well-chosen materials will saturate with preservative rapidly. After the material is saturated, preservative loss from drug product is driven by the rate of preservative diffusion through the material and evaporation into the surrounding environment. Because of this, preservative loss will be driven by residence time of drug product in a component, diffusion rate of preservative through materials, and material thickness.

What is needed, therefore, is an insulin delivery system that maintains insulin stability and/or prevents preservative loss prior to delivery of the insulin.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows an exemplary insulin delivery system.

FIG. 1B shows a close-up of the hub of the delivery system of FIG. 1A.

FIG. 2 shows a cross-section of multi-layer tubing of an insulin delivery system where one layer is a barrier layer.

FIG. 3 shows a cross-section of a barrel of an insulin delivery system where the housing has a barrier layer therein.

FIG. 4 shows a cross-section of multi-layer tubing of an insulin delivery system where one layer is a ballast.

FIG. 5 shows a cross-section of a barrel of an insulin delivery system where the housing further has a ballast therein.

FIGS. 6A-6C show an insulin delivery system with a venting element.

FIG. 7 shows a particulate trap.

FIG. 8 shows a cross-section of a barrel of an insulin delivery system where the septum has a barrier layer thereon.

FIGS. 9A and 9B show various views of an embodiment of a cannula of an insulin delivery system.

FIGS. 10A-10B show various views of an embodiment of a cannula of an insulin delivery system.

FIGS. 11A-11C show various views of an embodiment of a cannula of an insulin delivery system.

FIGS. 12A and 12B show various views of an embodiment of a cannula of an insulin delivery system.

FIG. 13 shows a false-color fluorescence image of an embodiment of an infusion set.

FIG. 14 shows data comparing particulate matter found in various substances.

FIGS. 15A and 15B show embodiments of a cannula of an insulin delivery system.

FIG. 16 shows an embodiment of a method for manufacturing an infusion cannula using a heat shrink process.

FIG. 17 shows an embodiment of a cannula comprising features configured to locate/capture/constrain a coil.

FIGS. 18A and 18B show embodiments of a coil within a cannula produced using different methods.

FIG. 19 shows an embodiment of a barrel of an infusion cannula.

FIGS. 20A and 20B show embodiments of features of a barrel configured to locate/align/capture/constrain a septum.

FIGS. 21A and 21B show embodiments of a mold for producing an infusion cannula and a molded cannula.

FIG. 22 shows a cross section of an embodiment of a coil reinforced cannula.

FIG. 23 shows a graph comparing flexibilities of differently manufactured coil reinforced cannulas.

FIG. 24 shows an embodiment of a mold for molding an infusion cannula.

FIGS. 25A-26B show embodiments of barrel features configured to capture and hole a septum in place.

FIGS. 27A and 27B show an embodiment of a septum.

### SUMMARY OF THE DISCLOSURE

In a first aspect, embodiments of an insulin delivery system are provided. The delivery system comprises a reservoir configured to hold an insulin medication therein; an infusion hub; tubing fluidically connecting the insulin reservoir and the infusion hub; a cannula configured to deliver the insulin medication to a patient; and an absorbent element positioned within the delivery system and in fluidic contact with the insulin medication, the absorbent element configured to absorb and store preservatives from the insulin medication.

In some embodiments, the system comprises an impermeable backing layer adjacent to the absorbent element and configured to maintain the preservatives within the absorbent.

The absorbent element can comprise EVOH, silicone, a low-density polymer, a PEG block-copolymer (e.g., PETG), PET, nylon, a nylon block-copolymer, a polymeric foam, or a polymeric monolith.

In some embodiments, the absorbent comprises a preservative capacity greater than a maximum concentration of preservative in the insulin medication.

The absorbent can be further configured to release preservatives to the insulin medication after storing the preservatives.

In some embodiments, the absorbent is configured to maintain the preservative concentration at the point of delivery to the patient at a concentration that minimizes local toxicity while maintaining insulin in a stable hexameric state.

The absorbent can be configured to maintain the preservative concentration at a concentration of greater than about 1.25 mg/mL. The absorbent can be configured to maintain the preservative concentration at a concentration of about 1.15-1.75 mg/mL. The absorbent can be configured to maintain the preservative concentration at a concentration of about 1.25-1.50 mg/mL.

In some embodiments, the absorbent comprises an interior layer of the tubing.

At least a portion of the insulin delivery system can be configured to prevent migration of preservatives from the insulin medication.

In some embodiments, the tubing is a multi-layer tubing, and wherein the barrier layer is at least a portion of a layer of the multi-layer tubing. The barrier layer can form an entire layer of the multi-layer tubing.

In some embodiments, the tubing comprises the barrier layer. The barrier layer can comprise a coating on the tubing. In some embodiments, the barrier layer comprises an inner layer of the tubing.

The barrier layer can comprise polyether block-amide, HDPE, polypropylene, PTFE, chloro- and fluorosilicones, hydrochloro-, hydrofluoro-, and perfluoro-polymers, chlorinated polymers (e.g., viton), metal-coated polymers (e.g., mylar), poly carbonate, organic or inorganic plasma-deposited coatings (e.g. PTFE, PVC, halogenated siloxanes, silicon suboxides), vapor-deposited coatings (such as nitrides, titanium nitride, fluorocarbons, metals), Kapton, or parylene.

In some embodiments, the system comprises a barrel connected to the cannula, and wherein the barrier layer is positioned on at least a portion of the barrel.

A connector can comprise the barrier layer.

In some embodiments, the barrier layer extends through an entire fluid path of the system. In a further aspect, embodiments of a method of delivering insulin medication are provided. The method comprises providing a delivery system; delivering insulin medication comprising preservatives from a reservoir through tubing connecting the reservoir to an infusion hub; delivering the insulin medication to the patient through a cannula connected to the infusion hub, wherein delivering the insulin medication comprises exposing the insulin medication to an absorbent element configured to absorb and store preservatives from the insulin medication. In some embodiments, the method comprises the absorbent element absorbing preservatives from the insulin medication. The method can comprise the absorbent element releasing preservatives back into the insulin medication.

In some embodiments, the method comprises preventing preservative evaporation using a barrier layer on one or more components of the delivery system.

In another aspect, embodiments of an insulin delivery system are provided. The system comprises a reservoir configured to hold an insulin medication therein; an infusion hub; tubing connecting the insulin reservoir and the infusion hub; a cannula configured to deliver the insulin to a patient; and a vent in the tubing or the hub configured to release preservatives from the insulin medication prior to delivery of the insulin medication to the patient.

In some embodiments, at least a portion of the insulin delivery system includes a barrier layer configured to prevent migration of preservatives from the insulin medication.

The vent can comprise an opening in the barrier layer. In some embodiments, the vent comprises a portion of the barrier layer that is thinner than other portions of the barrier layer. The vent can comprise a portion of the barrier layer that is thinner than surrounding portions of the barrier layer.

In some embodiments, the tubing is a multi-layer tubing, and wherein the barrier layer is at least a portion of a layer of the multi-layer tubing. In some embodiments, the tubing is a multi-layer tubing, and wherein the barrier layer forms an entire layer of the multi-layer tubing.

The tubing can comprise the barrier layer. In some embodiments, the barrier layer comprises a coating on the tubing. In some embodiments, the barrier layer comprises an inner layer of the tubing.

The barrier layer can comprise polyether block-amide, HDPE, polypropylene, PTFE, chloro- and fluorosilicones, hydrochloro-, hydrofluoro-, and perfluoro-polymers, chlorinated polymers (e.g., viton), metal-coated polymers (e.g., mylar), poly carbonate, organic or inorganic plasma-deposited coatings (e.g. PTFE, PVC, halogenated siloxanes, silicon suboxides), vapor-deposited coatings (such as nitrides, titanium nitride, fluorocarbons, metals), Kapton, or parylene.

In some embodiments, the system comprises a barrel connected to the cannula, and wherein the barrier layer is positioned on at least a portion of the barrel.

In some embodiments, a connector comprises the barrier layer.

The barrier layer can extend through an entire fluid path of the system.

In some embodiments, the vent comprises EVOH, silicone, a low-density polymer, a PEG block-copolymer (e.g., PETG), PET, nylon, or a nylon block-copolymer.

In some embodiments, the vent comprises an opening in a wall or layer of the tubing or the hub.

In yet another aspect, embodiments of a method of delivering insulin are provided. The method comprises providing a delivery system; delivering insulin medication comprising preservatives from a reservoir through tubing connecting the reservoir to an infusion hub; delivering the insulin medication to the patient through a cannula connected to the infusion hub; and venting the insulin medication, thereby releasing preservatives from the insulin medication.

In some embodiments, the method comprises preventing preservative loss from the insulin medication by providing a barrier layer along at least a portion of the fluid path of the delivery system. Venting the insulin medication can comprise exposing the insulin medication to an opening in the barrier layer. In some embodiments, venting the insulin medication comprises exposing the insulin medication to a portion of the barrier layer that is thinner than other portions of the barrier layer. In some embodiments, venting the insulin medication comprises exposing the insulin medication to a portion the barrier layer that is thinner than surrounding portions of the barrier layer.

In another aspect, embodiments of an insulin delivery system are provided. The system comprises a reservoir configured to hold an insulin medication therein; an infusion hub; tubing connecting the insulin reservoir and the infusion hub; a cannula configured to deliver the insulin medication to a patient; and a filter configured to capture particulates from the insulin medication prior to delivery of the insulin to the patient.

In some embodiments, the filter comprises features internal to the cannula and in fluidic contact with the insulin configured to affect hydrodynamic flow characteristics of insulin medication flowing within the cannula and to create pressure differential regimes to promote the capture and retention of aggregate particles that have formed out of solution.

The features can repeat along at least a portion of a length of the cannula.

In some embodiments, a ratio of a width of the features and a period of the features is greater than about 1:1 and less than about 1:4.

In some embodiments, the features comprise internally molded features within the cannula. In some embodiments, the features comprise internally extruded features within the cannula.

The internal features can comprise polyether block-amide.

In some embodiments, the filter comprises an internal coil within the cannula.

The internal coil can comprise a round wire. The internal wire can comprise a flat wire.

In some embodiments, the internal coil comprises an engineering polymer. In some embodiments, the internal coil comprises stainless steel.

In some embodiments, the filter comprises a structural component to prevent crushing or kinking of the extruded cannula.

The filter can comprise a threaded inner surface of the cannula.

The threaded surface can comprise angular or pointed threads. The threaded surface can comprise flat threads.

The threaded surface can comprise angled, overlapping, or buttress style threads. In some embodiments, at least a portion of the insulin delivery system includes a barrier layer configured to prevent migration of preservatives from the insulin medication.

The tubing can be a multi-layer tubing, and the barrier layer can be at least a portion of the multi-layer tubing. The tubing can be a multi-layer tubing, and the barrier layer can form an entire layer of the multi-layer tubing.

In some embodiments, the tubing comprises the barrier layer. The barrier layer can comprise a coating on the tubing. The barrier layer can comprise an inner layer of the tubing.

In some embodiments, the barrier layer comprises polyether block-amide, HDPE, polypropylene, PTFE, chloro- and fluorosilicones, hydrochloro-, hydrofluoro-, and perfluoro-polymers, chlorinated polymers (e.g., viton), metal-coated polymers (e.g., mylar), poly carbonate, organic or inorganic plasma-deposited coatings (e.g. PTFE, PVC, halogenated siloxanes, silicon suboxides), vapor-deposited coatings (such as nitrides, titanium nitride, fluorocarbons, metals), Kapton, or parylene.

The system can comprise a barrel connected to the cannula, and the barrier layer can be positioned on at least a portion of the barrel.

A connector can comprise the barrier layer.

In some embodiments, the barrier layer extends through an entire fluid path of the system. The system can comprise a vent in the tubing or the hub configured to release preservatives from the insulin medication prior to delivery of the insulin medication to the patient.

In some embodiments, at least a portion of the insulin delivery system includes a barrier layer configured to prevent migration of preservatives from the insulin medication and wherein the vent comprises an opening in the barrier layer. In some embodiments, at least a portion of the insulin delivery system includes a barrier layer configured to prevent migration of preservatives from the insulin medication and wherein the vent comprises a portion of the barrier layer that is thinner than other portions of the barrier layer. In some embodiments, at least a portion of the insulin delivery system includes a barrier layer configured to prevent migration of preservatives from the insulin medication and wherein the vent comprises a portion of the barrier layer that is thinner than surrounding portions of the barrier layer.

The system can further comprise an absorbent element positioned within the delivery system and in fluidic contact with the insulin medication, the absorbent element configured to absorb and store preservatives from the insulin medication.

The system can comprise an impermeable backing layer adjacent to the absorbent element and configured to maintain the preservatives within the absorbent.

In some embodiments, the absorbent element comprises EVOH, silicone, a low-density polymer, a PEG block-copolymer (e.g., PETG), PET, nylon, a nylon block-copolymer, a polymeric foam, or a polymeric monolith.

The absorbent can comprise a preservative capacity greater than a maximum concentration of preservative in the insulin medication.

In some embodiments, the absorbent is further configured to release preservatives to the insulin medication after storing the preservatives.

The absorbent can be configured to maintain the preservative concentration at the point of delivery to the patient at a concentration that minimizes local toxicity while maintaining insulin in a stable hexameric state.

The absorbent can comprise an interior layer of the tubing.

In still a further aspect, embodiments of a method for delivering insulin are provided. The method comprises providing a delivery system; delivering insulin medication comprising preservatives from a reservoir through tubing connecting the reservoir to an infusion hub; filtering the insulin medication to capture particulates from the insulin medication prior to delivery of the insulin medication to the patient; and delivering the insulin medication to the patient through a cannula connected to the infusion hub.

In some embodiments, the method comprises affecting hydrodynamic flow characteristics of insulin medication flowing within the cannula and creating differential pressure regimes to promote the capture and retention of aggregate particles that have formed out of solution.

Filtering the insulin medication can comprise providing, within a flow path of the insulin medication, features internal to the cannula, thereby affecting hydrodynamic flow characteristics of insulin medication flowing within the cannula and creating differential pressure regimes to promote the capture and retention of aggregate particles that have formed out of solution.

Filtering the insulin medication can comprise using internally molded features within the cannula. Filtering the insulin medication can comprise using internally extruded features within the cannula.

In some embodiments, filtering the insulin medication comprises using a coil internal to the cannula, the coil configured to create a region of features affecting hydrodynamic flow characteristics of insulin medication flowing within the cannula and creating differential pressure regimes to promote the capture and retention of aggregate particles that have formed out of solution.

The coil can comprise a round wire. The coil can comprise a flat wire.

The coil can comprise stainless steel. The coil can comprise an engineering polymer.

The method can further comprise exposing the insulin medication to an absorbent element configured to store preservatives from the insulin medication.

In some embodiments, the method comprises the absorbent element absorbing preservatives from the insulin medication.

The method can comprise the absorbent element releasing preservatives back into the insulin medication.

In some embodiments, the method comprises preventing preservative evaporation using a barrier layer on one or more components of the delivery system.

The method can comprise venting the insulin medication, thereby releasing preservatives from the insulin medication.

In some embodiments, the method comprises preventing preservative loss from the insulin medication by providing a barrier layer along at least a portion of the fluid path of the delivery system, and wherein venting the insulin medication comprises exposing the insulin medication to an opening in the barrier layer. In some embodiments, the method comprises preventing preservative loss from the insulin medication by providing a barrier layer along at least a portion of the fluid path of the delivery system, and wherein venting the insulin medication comprises exposing the insulin medication to a portion of the barrier layer that is thinner than other portions of the barrier layer. In some embodiments, the method comprises preventing preservative loss from the insulin medication by providing a barrier layer along at least a portion of the fluid path of the delivery system, and wherein venting the insulin medication comprises exposing the insulin medication to a portion of the barrier layer that is thinner than surrounding portions of the barrier layer.

In another aspect, embodiments of an insulin delivery system are provided. The system comprises a reservoir configured to hold an insulin medication therein; an infusion hub; tubing fluidically connecting the insulin reservoir and the infusion hub; and a cannula configured to deliver the insulin medication to a patient; wherein at least a portion of the insulin delivery system includes a barrier layer configured to prevent migration of preservatives from the insulin medication.

In some embodiments, the tubing is a multi-layer tubing, and wherein the barrier layer is a layer of the multi-layer tubing.

The barrier layer can comprise polyether block-amide, HDPE, polypropylene, PTFE, chloro- and fluorosilicones, hydrochloro-, hydrofluoro-, and perfluoro-polymers, chlorinated polymers (e.g., viton), metal-coated polymers (e.g., mylar), poly carbonate, organic or inorganic plasma-deposited coatings (e.g. PTFE, PVC, halogenated siloxanes, silicon suboxides), vapor-deposited coatings (such as nitrides, titanium nitride, fluorocarbons, metals), Kapton, or parylene.

### DETAILED DESCRIPTION

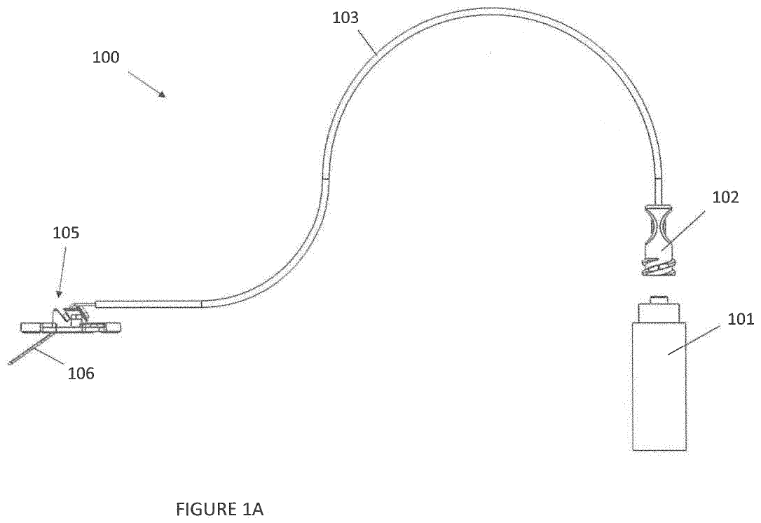

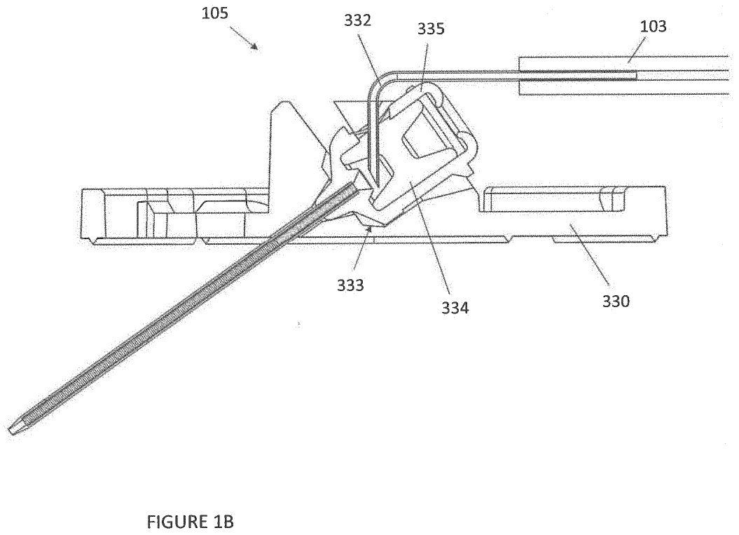

Referring to FIG. 1A, an insulin delivery system 100 includes an insulin reservoir 101 (configured to store an insulin medication therein), a reservoir-set connector 102, tubing 103, a hub 105, and an infusion cannula 106. As shown in FIG. 1B, the hub 105 can include a patch 330 or other mechanism configured to adhere to the patient, a barrel 333 connected to the cannula 106, and an introducer 332 extending from the tubing 103. The barrel 333 can include a mechanical housing 335 configured to house a septum. The fluid introducer 332 can be configured to pierce the septum 334 and to deliver fluid to the cannula 106. The fluid path for the insulin medication, therefore, can run from the reservoir 101 through the tubing 103 to the barrel 333 and to the patient via the cannula 106.

In some embodiments, some or all of the fluid path can include a barrier therein to inhibit preservative evaporation and/or loss. The barrier can be, for example, a coating or layer positioned along the fluid path. In other embodiments, the barrier can be the entire component (e.g., the entire the set-cannula connector 105). In some embodiments, each component can have a different barrier.

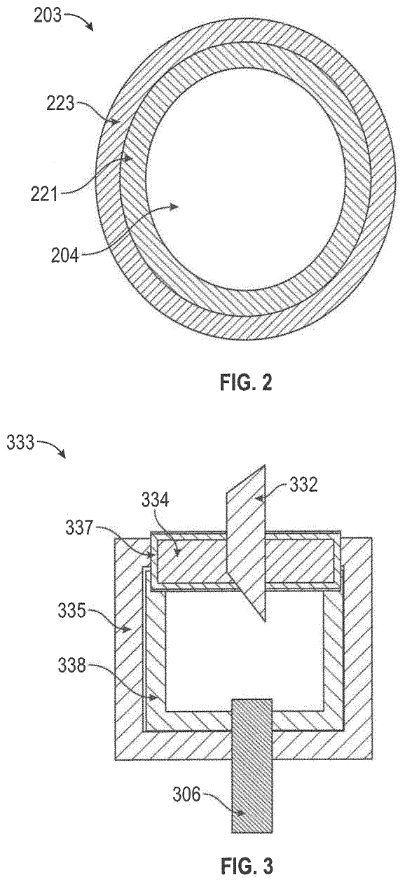

For example, referring to FIG. 2, the tubing 203 can be a multi-layer tubing, wherein the fluid path 204 is surrounded by a barrier 221 configured to prevent preservative loss and an outer layer 223 configured to provide mechanical protection and/or bond to other components of the system.

As another example, referring to FIG. 3, the septum 334 can be coated with a barrier 337 to minimize preservative loss and/or the interior of the mechanical housing 335 can be lined with a barrier 338 to minimize preservative loss. In some embodiments, shown in FIG. 8, the barrier 337 can be on only one side of the septum 334.

In embodiments where the barrier extends along multiple components, the barrier can be discrete or continuous. Similarly, the barrier can have different characteristics (e.g., be made of different materials) from component to component or can be the same from component to component.

The barriers described herein can include, for example, polyether block-amide, HDPE, polypropylene, PTFE, chloro- and fluorosilicones, hydrochloro-, hydrofluoro-, and perfluoro-polymers, chlorinated polymers (e.g. viton), metal-coated polymers (e.g., mylar), poly carbonate, organic or inorganic plasma-deposited coatings (e.g. PTFE, PVC, halogenated siloxanes, silicon suboxides), vapor-deposited coatings (such as nitrides, titanium nitride, fluorocarbons, metals), Kapton, or parylene.

In some specific examples, a length of the tubing 332 within the barrel 333 can prevent preservative evaporation. The outer housing 335 of the barrel 333 can also serve as a barrier if some of the internal fluid-contacting components need to be made from specific materials because of their physical/mechanical properties. In another embodiment, a connector (e.g., connector 102) that includes a fluid path may be fabricated from ceramic, or polytetrafluoroethylene or another a material with low permeability to preservative. In another embodiment, a barrier layer may form a continuous path that extends through more than one component in the system, such as a tube that originates at the connector 102 and extends through the tubing 332, the hub 105, and ends with a direct connection to the cannula 106.

In some embodiments, the fluid path can include a ballast therein. The ballast can be an absorbent material configured to absorb and release preservatives from the insulin medication. The ballast can advantageously be configured to absorb preservatives from the insulin medication when there is a high preservative concentration (e.g., when the insulin medication moves quickly during bolus delivery or priming) and to release preservatives into to the insulin medication when the preservative concentration is low (e.g., when the drug product moves slowly during basal delivery or when delivery is suspended or stopped).

The ballast can help maintain the preservative concentration at the point of delivery to the patient at a concentration that minimizes local toxicity while maintaining insulin in a stable hexameric state. In some embodiments this concentration can be about greater than 1.25 mg/mL (or 1.25-1.50 mg/mL or 1.15-1.75 mg/mL, etc.). The ballast can thus create a “smoothed” preservative concentration vs. time profile, which can advantageously provide: (1) a consistent pharmacokinetic profile resulting from consistent preservative concentration at the point of delivery because preservative absorption into tissue is a step that must occur before insulin can be absorbed; and (2) a decrease in the incidence of site loss because maintaining preservative concentration above the threshold needed for insulin stability will reduce the amount of insulin aggregates introduced at the infusion site.

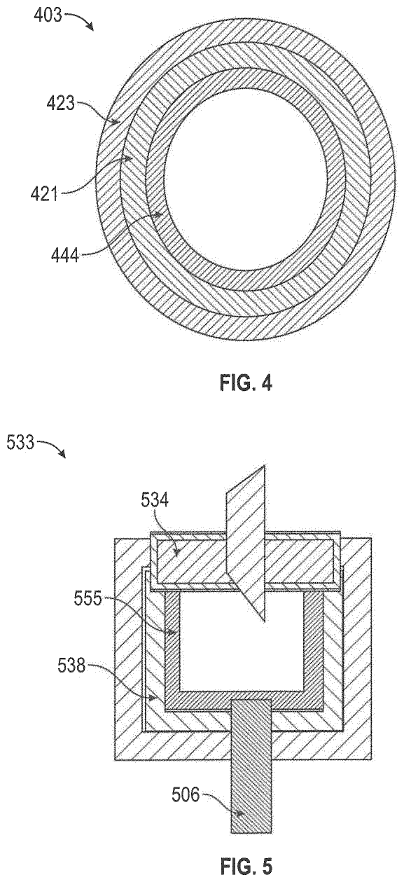

For example, referring to FIG. 4, a ballast 444 can form an interior layer of the tubing 403. The ballast 444 can be bordered by a barrier 421 to prevent the preservatives from leaving the ballast 444. Outer layer 423 can surround the barrier 421, similar to as described with respect to tubing 203.

As another example, referring to FIG. 5, a ballast 555 can form an interior layer of the housing 335 of a barrel 533. A barrier layer 538 can border the ballast 555 to prevent the preservatives from leaving the ballast 555.

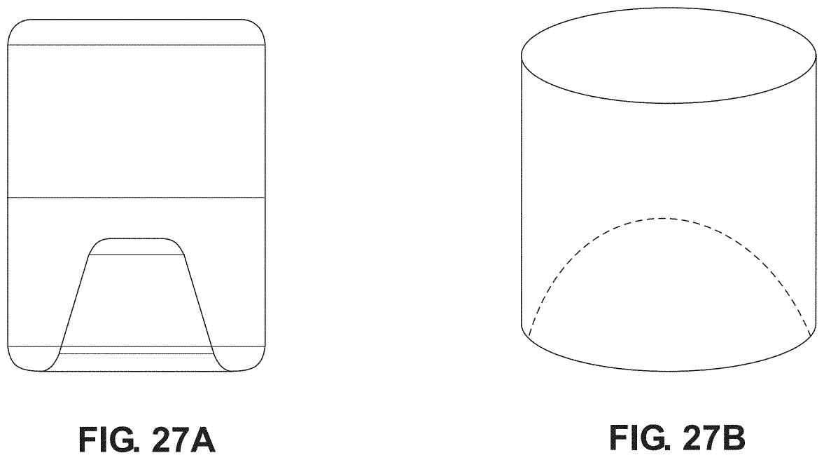

In some embodiments, the septum (FIG. 27A) comprises an absorbent element, as. The septum can be configured to absorb and store preservatives from the insulin medication.

In some embodiments, this component functions first as an absorbent element positioned within the delivery system, in fluidic contact with the insulin medication, wherein the absorbent element is configured to absorb and store preservatives from the insulin medication and secondarily in the traditional use of a “septum” wherein the component acts as a sterile barrier and one or more needles or canullae pierce the septum to connect discreet elements of a fluidic system.

In some embodiments, the septum can be modified from the design shown in FIG. 27A by adding additional material (e.g., silicone). FIG. 27B shows an embodiment of such a septum design. In some embodiments, about 2.5-3.5 mm³ additional material can be added (e.g., about 2.8 mm³). This modification may help increase the absorbent properties of the septum.

The modified septum can have a top-side surface area of about 4.5 mm² (or about 4-5 mm²).

In some embodiments, the system comprises a selective or semi-permeable barrier layer adjacent to the absorbent element and configured to maintain the preservatives within the absorbent.

In some embodiments the barrier layer is the body of barrel.

The absorbent element can comprise EVOH, silicone, a low-density polymer, a PEG block-copolymer (e.g., PETG), PET, nylon, a nylon block-copolymer, a polymeric foam, or a polymeric monolith.

The ballast(s) can advantageously be configured to maintain a consistent preservative concentration at the point of delivery (e.g., from the cannula 506 to the patient) by acting as a damper or sink for preservative concentration. The ballast can advantageously counteract the effects of insulin medication passing through portions of the fluid path (e.g., the septum 534) where preservative evaporation cannot be prevented.

The ballast can advantageously work in the following non-limiting manner.

The preservative concentration of any given unit of insulin delivered by a continuous subcutaneous insulin infusion set (e.g., the infusion delivery system described herein) is a function of the residence time of that unit within the fluid path components were preservative evaporation occurs. When a bolus is delivered, the residence time of the fluid is short—and hence preservative loss is small and preservative concentration remains near the initial level. When the system is delivering at basal rates of when delivery is temporarily stopped, the residence time of a unit of insulin medication is longer, and therefore there is more time for preservative to be lost by diffusion/evaporation at one or more elements within the fluid path.

In some embodiments, the priming process can serve to charge the ballast. When the insulin delivery system is primed (e.g., prior to use), the insulin medication moving through the system can have the minimum possible residence time, and therefore the highest possible preservative concentration. If a preservative ballast is in this fluid path, it can absorb some of the preservative from the initial bolus. This can load the ballast up with preservative. In some embodiments, the effect can be to reduce the initial concentration of preservative and possibly reduce the toxicity of the initial fluid delivered to the patient by reducing the initial preservative exposure.

Additionally, bolus delivery is much faster than basal and can be as fast as priming. Accordingly, bolus delivery can offer another opportunity to charge the ballast (similar to as described with respect to priming).

During basal delivery or when delivery is stopped temporarily, the ballast can return preservative to the insulin flowing past. In the case of basal insulin delivery, the insulin reaching the point of delivery is generally preservative-depleted. This situation is the hardest on delivered insulin because (1) the preservative stabilization effect is at its lowest and (2) the insulin spends the most time in a preservative-depleted environment where aggregation is most favored. The ballast can advantageously release preservative into preservative-depleted drug product that passes by. This can increase the insulin stabilization. In some embodiments, the preservative ballast can be positioned directly downstream of any known point of preservative loss, such as just downstream of turns or angles in the fluid path or areas where the infusion set tubing does not adequately prevent preservative loss.

The ballast can be made of a material that enables preservative to diffuse rapidly, but does not cause the preservative to be sequestered irreversibly. Exemplary materials for the ballast include polymers with highly disordered domains or materials with aromatic groups that can act to solvate preservative molecules within the preservative matrix. For example, the ballast can be made of EVOH, silicone, a low-density polymer, a PEG block-copolymer (e.g., PETG), PET, nylon, a nylon block-copolymer, polyurethane, or polyurethane copolymers.

### Example 1

Approximately 0.5 g of four different candidate materials were chopped into small pieces with edges generally no larger than about 4 mm² with a clean razor. The material was transferred into a clean, tared 4 mL vial and weighed to determine the actual mass of material. Each vial was filled with 3 mL of 20 mg/mL m-cresol solution, sealed, and agitated at ambient temperature for 2 hours. At the end of this time, the m-cresol solution was decanted and each solid was quickly washed 3 times with 2 mL DI water (<5 sec per wash with mixing). After the final wash, residual water was decanted to the greatest extent possible. Finally, each material sample was covered with fresh DI water and left static at room temperature for 1 hour. (Note that the residence time of insulin U100 within an infusion set can be up to 8 hours.) The resulting solutions were decanted and transferred into tared HPLC vials. The mass of each extract solution was determined. 85% recovery of the extraction fluid was assumed for calculations. The amount of m-cresol in each extract was determined with HPLC. The results are provided in Table 1, below:

TABLE 1

| | [m- | | | Approx. |
|---|---|---|---|---|
| | cresol] | Mass | Mass | Cap. = |
| Material | mg/g | Solid | Water | mg/g |
| Pebax 7233 | 3.16 | 469 | 665 | 5.27 |
| Pebax 6333 | 2.41 | 447 | 740 | 4.70 |
| Silibione | 3.13 | 446 | 875 | 7.23 |
| 4745 | | | | |
| Silibione | 4.34 | 442 | 625 | 7.22 |
| 4747 | | | | |

These results show that the preservative capacity of all these materials exceeds the maximum preservative concentration in the most common rapid-acting insulin U-100 products used for CSII therapy, such as 3.22 mg/mL total for insulin aspart products (e.g., Novolog, Fiasp) and 3.15 mg/mL total for insulin lispro products (e.g. Humalog, Lyumjev, Admelog).

Incubation of an excess of fluid with each material brought the preservative concentration from zero up to a level above the minimum required (>1.15 mg/mL) to maintain antimicrobial effectiveness in insulin U-100 drug products.

It will be appreciated that the embodiments of ballasts described herein are not limited to those embodiments described in Example 1.

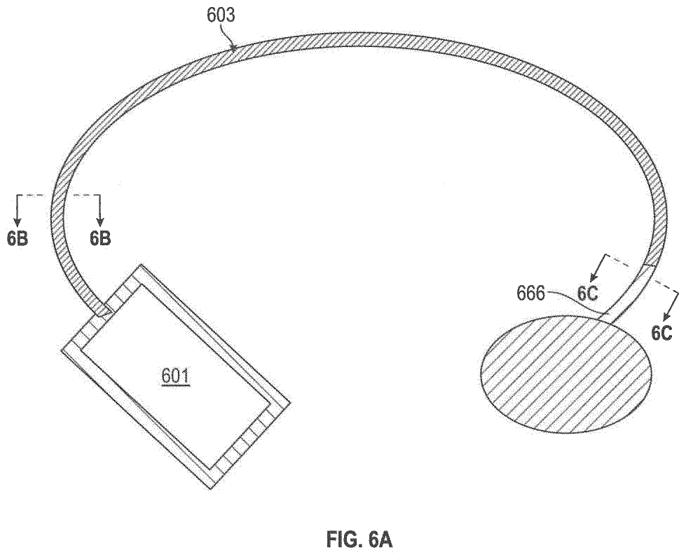

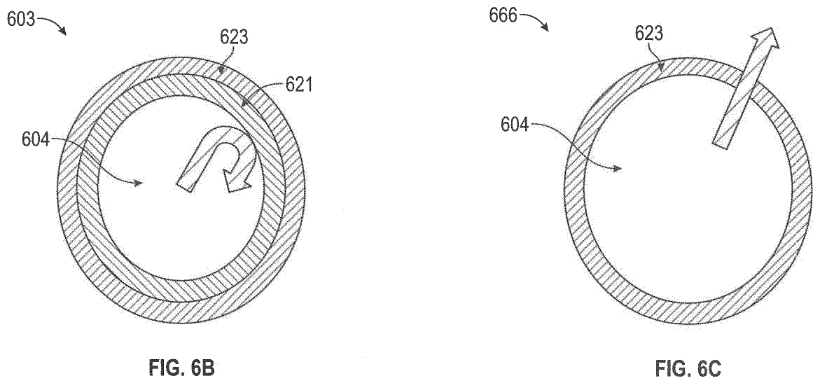

In some embodiments, the insulin delivery system can include a venting element therein configured to release excess preservatives just prior to delivery of insulin to the patient (e.g., so as to reduce inflammation or other reaction in the patient due to the inherent toxicity of the preservatives). For example, referring to FIGS. 6A-6C, the insulin delivery system can include a barrier 621 radially inwards of the outer wall 623 of the tubing 603. The barrier 621 can end, however, at a vent 666 that is adjacent and/or proximate to the hub. The vent 666 can thus be positioned along the fluid path 604 from the reservoir 601 just prior to delivery of insulin to the patient. As shown by the arrow in FIG. 6B, the barrier 621 can prevent preservative from leaving the fluid path 604. However, as shown by the arrow in FIG. 6C, the vent 666 (i.e., portion without the barrier 621) can allow the preservative to leave the fluid path 604. The vent 666 can thus allow preservative to exit the system prior to being delivered to the patient.

In some embodiments, the vent 666 can be an opening (e.g., an annular opening). In other embodiments, the vent 666 can include a thinning in the barrier, a material with lower barrier properties, a perforation of the barrier, or a material that has a high degree of preservative attraction in contact with the fluid path and the exterior environment. In some embodiments, for example, the vent 666 can be made of EVOH, silicone, a low-density polymer, a PEG block-copolymer (e.g., PETG), PET, nylon, or a nylon block-copolymer.

In some embodiments, the vent 666 can be positioned inside the hub rather than proximal or adjacent to the hub.

In some embodiments, the insulin delivery system described herein can include a filter configured to capture particulates (e.g., aggregated insulins or lubricating oils) from the insulin prior to delivery to the patient.

In some embodiments the filter can comprise features internal to at least a portion of the delivery system (e.g., the cannula) that affect hydrodynamic flow characteristics of insulin medication flowing within the cannula and create differential pressure regimes to promote the capture and retention of aggregate particles that have formed out of solution.

In some embodiments, the internal features comprise repeating mechanical features that are configured to form differential pressure regimes within the flow of the medication. The mechanical features may comprise angular features, undulations, features with both angular and curved portions, etc.

In some embodiments, the spacing between the repeating features is configured to promote differential pressure regimes. The ratio of the width of each feature and the spacing between the repeating mechanical features can be about 1:1 to about 1:5 (or 1:1 to 1:2, or 1:1 to 1:3, or 1:1 to 1:4, or 1:1 to 1:6 or more, etc.).

In some embodiments, the internal features are formed through molding or extrusion (e.g., threads). Such features can provide a manufacturing advantage over, for example, a separate structure (e.g., coil) added to infusion set components as such features can be more inexpensive and easier to manufacture. Internal features formed through molding or extrusion also can provide sufficient column strength to a component such as a cannula to allow for insertion. Internal features formed through molding or extrusion can also provide sufficient modulus strength to the component while still providing enough flexibility to avoid tissue disruption.

In some embodiments, the internal features comprise polyether block-amide.

In some embodiments, the internal features are formed integrally with the infusion set components (e.g., cannula). In some embodiments, the internal features are separate components that are added to the infusion set components.

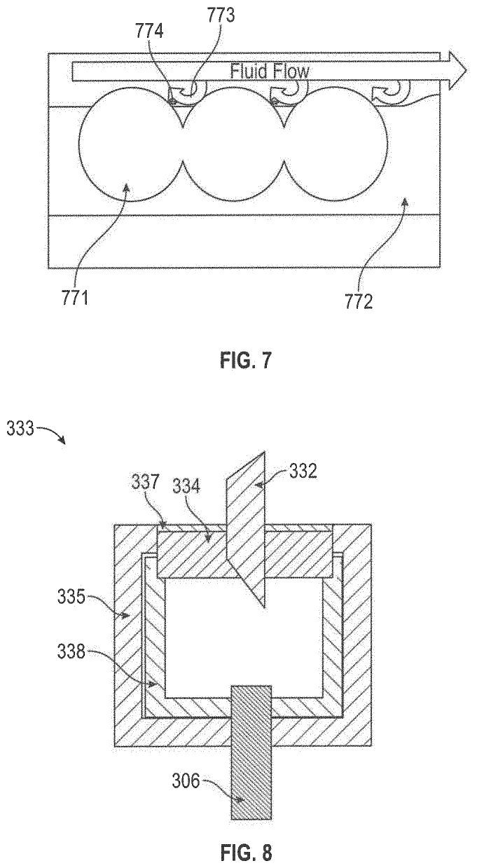

Referring to FIG. 7, a particulate trap in the cannula (e.g., along the length of an inner wall of the cannula) can include a metal coil 771 therein designed to provide mechanical flow resistance zones and cavities on the fluid-path periphery where large molecular assemblies (aggregates) become trapped while smaller molecules, such as insulin hexamers can diffuse back into the moving fluid.

The coil can comprise stainless steel or an engineering polymer.

The coil can comprise a round wire or a flat wire.

In some embodiments, the coil can be tightly wound with a pitch of 1:1 thereby creating a proliferation of differential pressure areas along the wall of the lumen created by the coil.

In other embodiments, the coil can be tightly wound with a pitch greater than 1:1 such as 1:2, 1:3 etc. thereby creating an extended differential pressure areas along the wall of the lumen created by the coil.

In some embodiments, the trap in the cannula can also include a high-friction inner tubing surface 772, where the surface finish can be controlled to have cavities that create small eddies 773 in the flow where large molecules 774 become trapped, but small molecules can diffuse out.

In some embodiments, the coil-reinforced cannula can be manufactured using a heat shrink assembly. The process comprises four unique steps: molding a pre-form; expanding a pre-form; inserting additional components; and heat shrinking the pre-form.

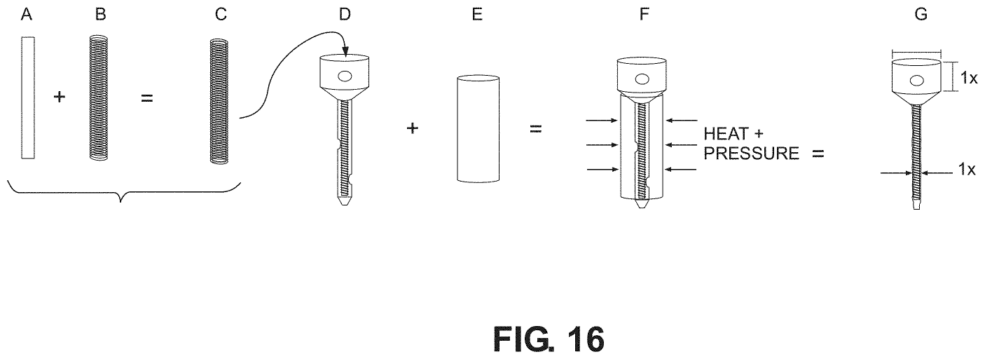

As shown in FIGS. 16A-16G, the heat shrinking process can comprise the following steps and components. The coil of FIG. 16B can be inserted over the mandrel of FIG. 16A creating the coil and mandrel assembly shown in FIG. 16C. The coil and mandrel assembly can be inserted into the molded cannula and barrel assembly, as shown in FIG. 16D. A heat shrink tubing, shown in FIG. 16E can be inserted over the cannula and barrel assembly of FIG. 16D. Heat and pressure can be applied to the heat shrink tubing causing the cannula body to press over the exposed coil, as shown in FIG. 16F. The final assembly is shown in FIG. 16G. In the final assembly, a wall thickness of the cannula can be the same as it was prior to the heat shrinking process. Other configurations are also contemplated.

In all the above disclosed mold production descriptions, the mold is designed to produce a component, herein called the pre-form, that, when irradiated and expanded, is capable of increasing all dimensions equally thereby preserving the aspect ratio of the finished part once heat shrunk. Additionally, the molded component is designed to shrink only to the smallest dimension molded when exposed to a temperature sufficient to initiate a relaxation of the imparted stress induced during the irradiation expansion process and returning the pre-form to its initial formative size. In order to produce an expanded component, the previously molded component is heated to just above the polymer's crystalline melting point and expanded in diameter, often by placing it in a vacuum chamber. While in the expanded state, it is rapidly cooled.

In some embodiments, the mold is designed to produce the cannula body only.

In these and other embodiments, the coil is inserted into the expanded pre-form and exposed to a temperature sufficient to initiate a relaxation of the imparted stress induced during the irradiation expansion process and returning the pre-form to its initial formative size (internal diameter(s)) and capturing the inserted components into pre-defined locations within the pre-form.

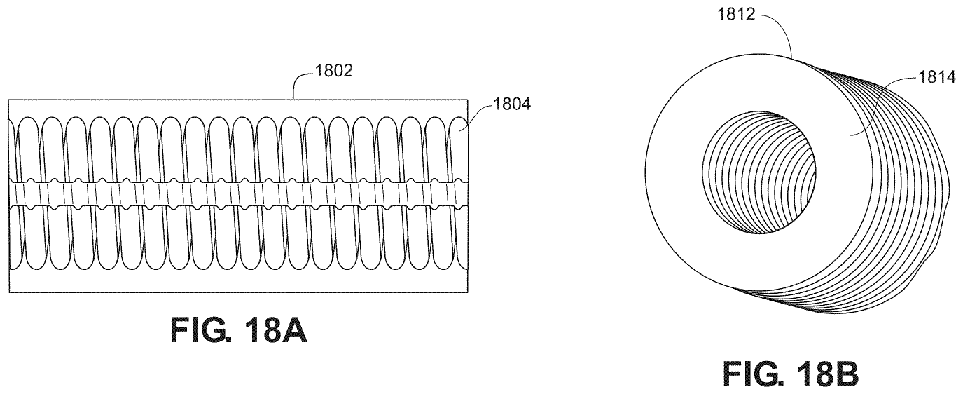

FIG. 18A shows a detailed view of the finished coil-cannula assembly. As shown in this figure the cannula portion 1802 intrudes minimally into the coil assembly 1804. In some embodiments, greater than 95% of the coil thickness remains exposed (or greater than 97%, greater than 99%, greater than 30%, greater than 50%, greater than 60%, greater than 75%, greater than 85%, etc.). The coil thickness that remains exposed can refer to the portion of the coil not embedded within the cannula wall. It is this portion of the coil that can form repeating mechanical features that are configured to form differential pressure regimes within the flow of the medication. FIG. 18B shows a detailed view of a coil-cannula assembly manufactured using traditional heat shrinking techniques. As shown in FIG. 18B, the cannula portion 1812 has intruded into/invaded the entire coil portion 1814, completely embedding it within the cannula portion 1812.

In these and other embodiments, as well as other embodiments, the pre-form has features unique to the design that locate and/or align the secondary component, or components, in a specific and fixed location and/or position.

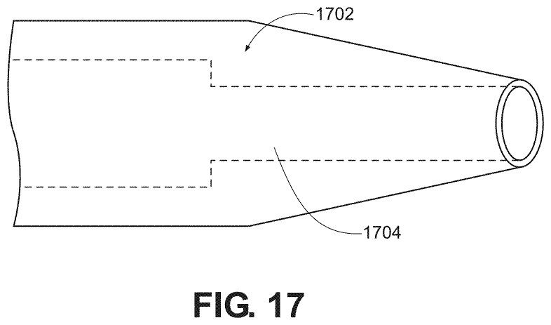

FIG. 17 shows an embodiment of a pre-form of a cannula comprising a shoulder 1702 configured to help located the inserted coil. The diameter of the coil can be greater than the diameter of the portion 1704 distal to the shoulder 1702, preventing the coil from extending past the shoulder 1702.

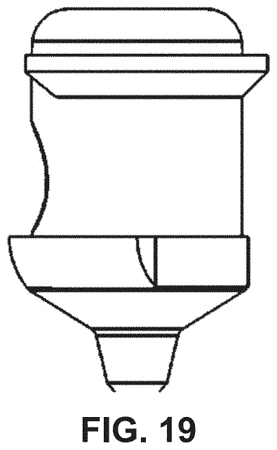

In some embodiments, a mold is designed to produce the barrel portion only, as shown in FIG. 19.

In these and other embodiments, the septum is inserted into the expanded pre-form and exposed to a temperature sufficient to initiate a relaxation of the imparted stress induced during the irradiation expansion process and returning the pre-form to its initial formative size (internal diameter(s)) and capturing the inserted components into pre-defined locations within the pre-form.

In these and other embodiments, the pre-form has features unique to the design that locate and/or align, or constrain/capture the secondary component, or components, in a specific and fixed location and/or position.

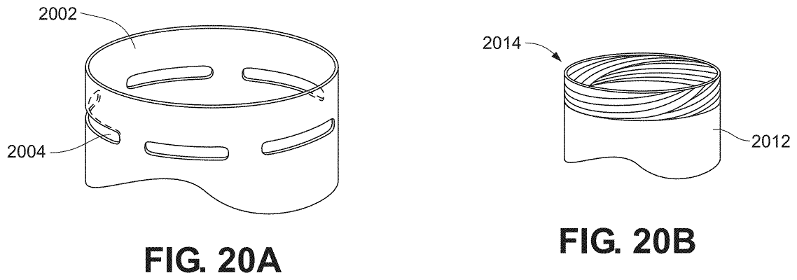

FIGS. 20A and 20B show embodiments of a barrel preform comprising features configured to locate and/or constrain/capture the septum. FIG. 20A shows an embodiment of a barrel 2002 comprising slits or grooves 2004 configured to locate/align/capture/constrain the septum. The heat shrink process can be configured such that the barrel ‘over-shrinks’ to capture the septum. FIG. 20B shows another embodiment of a barrel 2012 comprising thread features 2014 configured to allow septum capture.

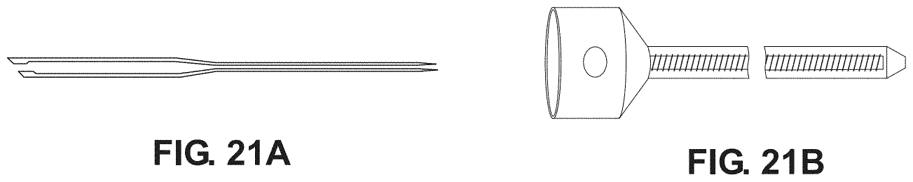

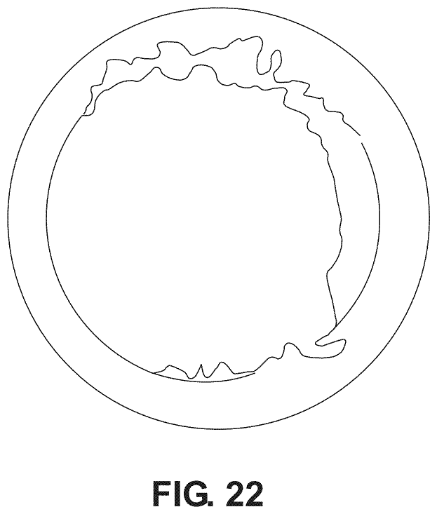

In some embodiments, the mold can be designed to produce the complete cannula and barrel body, as shown in FIG. 21A. As shown in FIG. 21B, in these and other embodiments, the septum and coil are inserted into the expanded pre-form and exposed to a temperature sufficient to initiate a relaxation of the imparted stress induced during the irradiation expansion process and returning the pre-form to its initial formative size (internal diameter(s) and capturing the inserted components into pre-defined locations within the pre-form.

In these and other embodiments, the pre-form has features unique to the design that locate and/or align, or constrain/capture the secondary component, or components, in a specific and fixed location and/or position.

It is advantageous to the shrink tube assembly process that the pre-form mold produces a component that only returns to its initial formative size (e.g., internal diameter(s)) after shrink. This advantage is not intuitive or currently taught. To the contrary, current methods, such as those described in U.S. patent Ser. No. 11/052,227B2 to Tegg does not describe the need or requirement for such a controlled molding process but rather the use of shrink tubing to tightly constrain or hold other components, a process that does not require a controlled shrink dimension.

It is advantageous that, in some embodiments, the shrink tube portion covering the coil and thereby forming the actual cannula section does not intrude or invade into the coil turns. As described herein, this configuration improves filtering capabilities and flexibilities of the cannula.

In the above described process of extrusion over the coil the polymer can also not intrude in the coil, as shown in FIG. 22, which shows a cross-section of an extruded cannula (polymer over coil).

There are non-obvious advantages in this assembly process. First, the polymer skin or over-jacket does not become a significant structural element in that is does not impede or constrain the coils from bending and stretching when impinged by external forces. Second, the internal diameter of the coil forms the primary fluid lumen which, in the laminar flow regime created by moving fluid, thereby creating areas of underpressure between the coils (pitch or lead) which act to promote capture and/or aggregation of precipitate or aggregate matter carried in suspension in the moving fluid. The exposed coils form riffles that filter the moving fluid (see (PCT/US 2021/048015, incorporated by reference herein).

In embodiments comprising a polymer over-jacket, the difference between unconstrained coils and completely constrained coils results in a measurable difference in deformation modulus which translates to a patient of user as flexibility and softness.

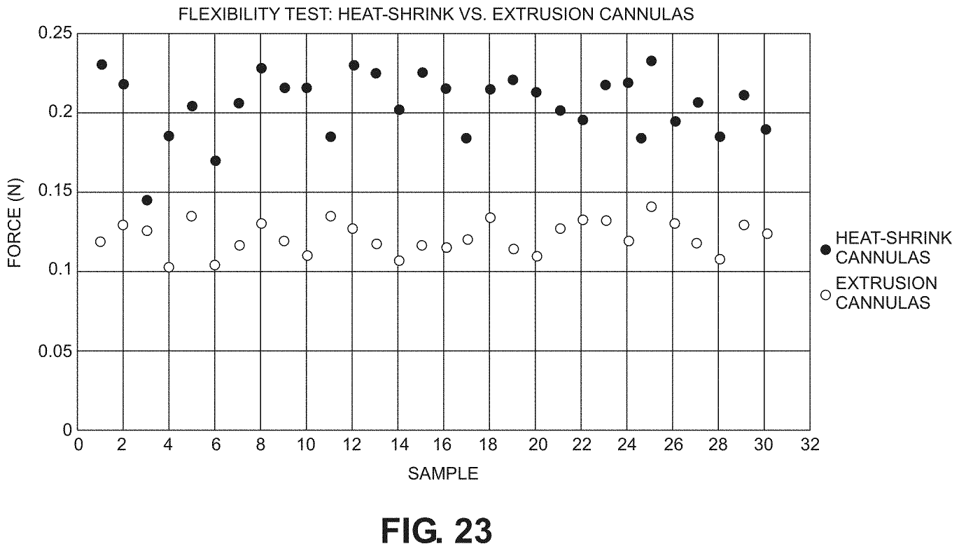

FIG. 23 shows a graph showing the difference in flexibility between a heat shrink cannula that allows polymer intrusion between the coils (FIG. 18B) and an extruded cannula that uses the internal structure created by the coils to support a skin of polymer.

In some embodiments, the filter comprises a threaded or similar surface within a portion of the delivery system or infusion set (e.g., the cannula). Any thread or other surface pattern that provides a series of repeating mechanical structures that promote differential pressure regimes within the flow path can be used.

The threaded inner surface may be formed via molding or extrusion.

The threaded inner surface may be formed integrally with the infusion or delivery component (e.g., cannula).

The threads may comprise sharp or pointed edges. In some embodiments, the threads comprise flat or square edges.

The threads may comprise a thread angle of about 0°-30°.

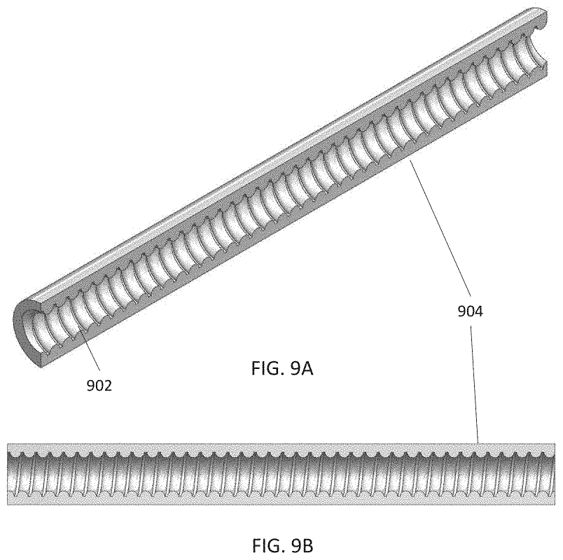

For example, in some embodiments, filter comprises a threaded surface 902 within the cannula 904 as shown in the perspective and side sectional views of FIGS. 9A and 9B. The threads may comprise angular, sharp, or pointed edges, as shown in FIG. 9A.

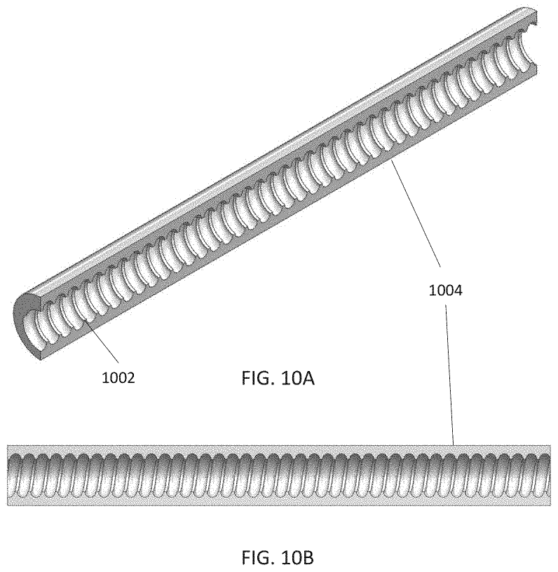

As described above, in some embodiments, the filter comprises a threaded surface 1002 within the cannula 1004, as shown in the perspective and side sectional views of FIGS. 10A and 10B. The threads may comprise flat edges. Such flat edges create increased differential pressure along the flat surface and thereby create increased negative pressure in the areas of the minor thread diameter (root).

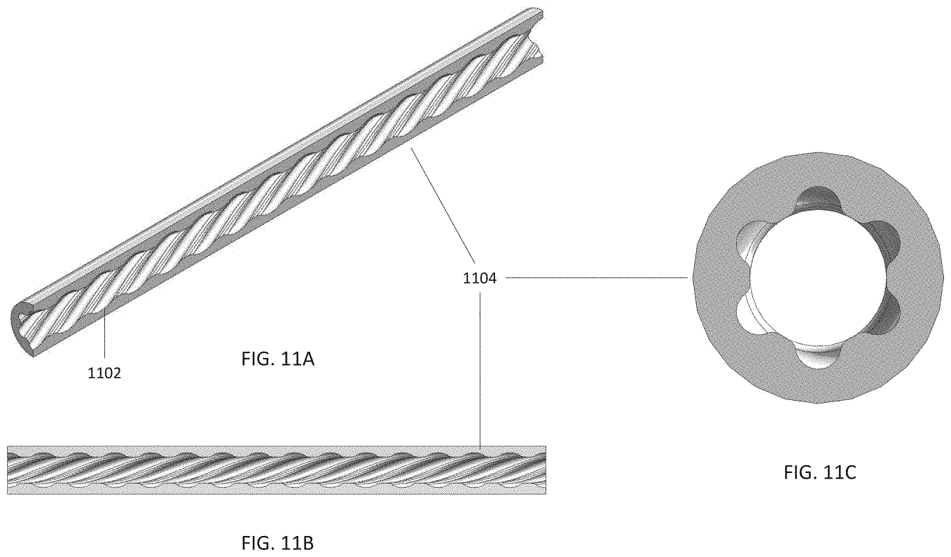

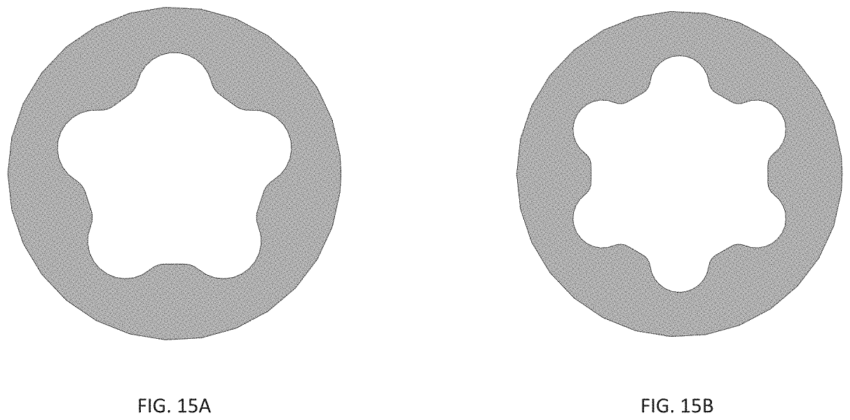

In some embodiments, the filter comprises a threaded surface 1102 within the cannula 1104 comprising a larger thread angle, as shown in the perspective and side sectional views of FIGS. 11A and 11B. For example, the thread angle can be about 30-75° (or about between 0-90°, 20-80°, 35-70°, 45-65°, etc.). A greater angle of the repeating structures (e.g., thread angle) can reduce the frequency of the repeating mechanical structure along the flow path of the medication. In certain such embodiments, the component may comprise a greater number of repeating angles, undulations, or other features affecting hydrodynamic flow characteristics of insulin medication flowing within the cannula and creating differential pressure regimes along a circumferential dimension, as shown in the end view of FIG. 11C. FIGS. 15A and 15B show other embodiments of a cannula with features affecting hydrodynamic flow characteristics of insulin medication flowing within the cannula and creating differential pressure regimes along a circumferential dimension of a cannula.

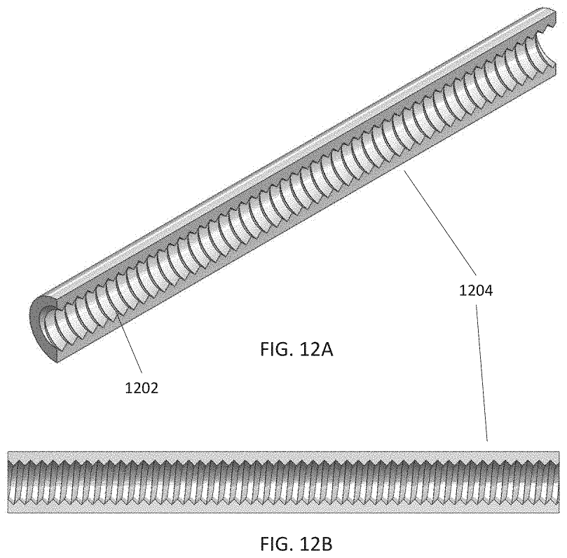

In some embodiments, the filter comprises a threaded surface 1202 within the cannula 1204, as shown in the perspective and side sectional views of FIGS. 12A and 12B. The threads may comprise angled, overlapping, or buttress style threads.

### Example 2

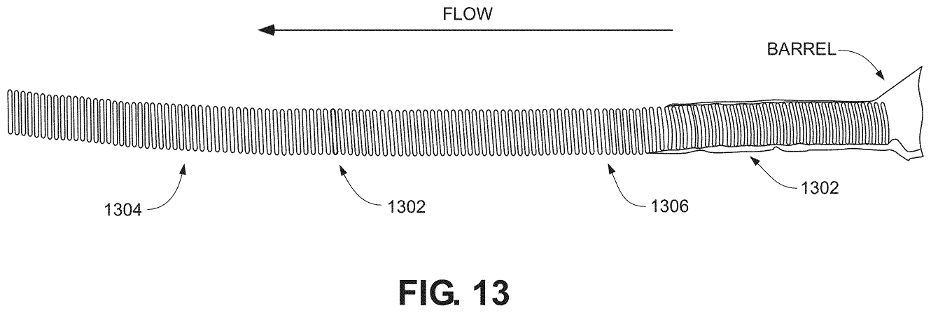

A clinical infusion set was flushed with Thioflavin-t solution. FIG. 13 shows a false-color fluorescence image of the infusion set. Black areas 1302 indicate fluorescence, and white areas 1304 show non-fluorescence. The infusion set comprises a stainless steel coil, visible as white stripes running the length of the infusion set 1306 (e.g., cannula). The black stripes show the insulin aggregates. This image demonstrates the efficacy of using a filter, as described herein to filter out aggregate particles from delivered medication.

### Example 3

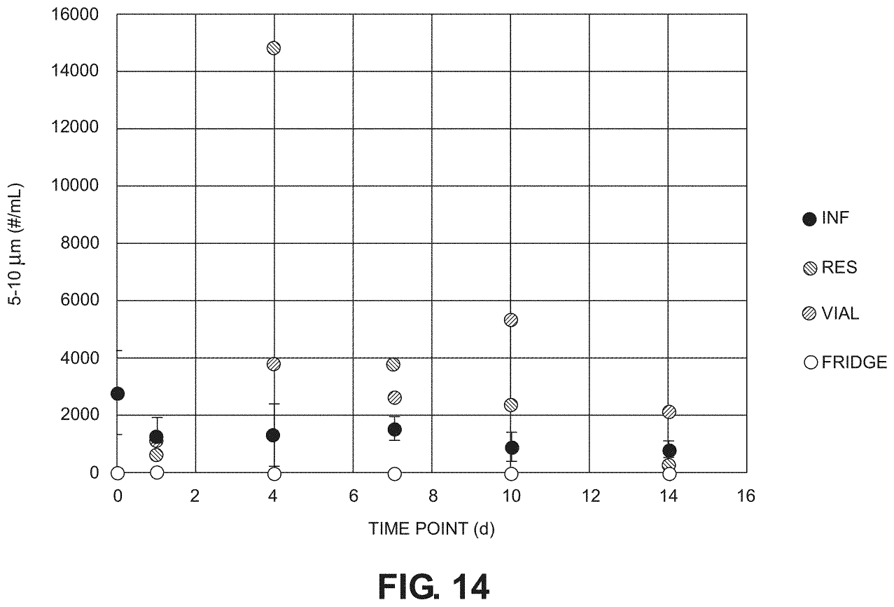

FIG. 14 shows data showing particulate matter collected from undelivered medication 1302, medication from a reservoir of a delivery system as described herein 1304, infusate medication 1306, and a control 1308. As shown in this figure, the infusate 1306 has much lower levels of particulate aggregation than the medication from the reservoir 1304, again demonstrating the efficacy of the filtering described herein.

In other embodiments, the particulate trap can include a non-circular lumen in the insulin delivery system with high-flow-resistance zones, such as star shape with acute-angle corners, configured so that the particulates collect in the resistance zones.

In other embodiments, the particulate trap can include features in the fluid path that cause mixing (mildly turbulent flow) near the center of the flow channel will facilitate particulate capture at the edges.

In other embodiments, the particulate trap can include a dead-end flow path (e.g., with preservative barrier properties) where non-dissolved particulate material will accumulate. In other embodiments, the particulate trap can include an inner surface of the fluid path of the insulin delivery system that is configured to specifically adhere silicone oil droplets, which can, in turn, capture insulin fibrils.

In other embodiments, the particulate trap can include a serpentine flow path, such as made of cured-in-place foam or open-cell foam lumen.

In other embodiments, the particulate trap can include lengthwise fibers threaded into the lumen where capture properties are included on the fibers (surface finish, chemistry, combination). The fibers, for example, can be pulled through the lumen using pressure differential across the length of the tubing.

In some embodiments, the particulate trap can include a flow path with high surface area.

In other embodiments, the particulate trap can include a conical cavity built into the flow path with flow dynamics designed to achieve cyclonic separation of particulates from dissolved material (e.g., such that captured particulates are directed into a sink at the tip of the cone).

In other embodiments, the particulate trap can include an affinity surface (e.g., hydrophobic, adhesive, fibril antibodies, aptamers, molecular templates) generated by chemical manipulation after extrusion. In other embodiments, the particulate trap can include a filler material in an inner lumen (e.g., carbon black, carbon tubules, ceramic nanoparticles) that protrude into the fluid path and acts as a trap for large molecular species, but permit the passage of insulin hexamer and smaller molecules.

In other embodiments, the particulate trap can include a highly hydrophobic inner surface. In other embodiments, the particulate trap can be part of a dual-lumen infusion set with a filtration membrane between the lumens (e.g., a hollow fiber filter within a larger tubing).

An exemplary method of producing the infusion set follows. A molded pre-form component is created. There are one or more distinct diameters on the pre-form. The pre-form is irradiated to expand the structure to a predefined ratio. One or more components are inserted into the pre-form. The assembly is exposed to a temperature sufficient to initiate a relaxation of the imparted stress induced during the irradiation expansion process and returning the pre-form to its initial formative size (internal diameter(s) and capturing the inserted components into pre-defined locations within the pre-form.

In this and other embodiments, the inserted components form the entire assembly when inserted into the pre-form. When exposed to a temperature sufficient to initiate a relaxation of the imparted stress induced during the irradiation expansion process and returning the pre-form to its initial formative size, the inserted coil (e.g., FIG. 18A) becomes a structural element in the assembly and limits the shrink of the pre-form to the external diameter of the coil. In this and other embodiments the coil is made from a metal or a polymer. The septum is captured by features previously disclosed and constrained within the design space only to the limit of the initial formative size. In this and other embodiments the septum is made from an elastomeric material suitable for drug delivery.

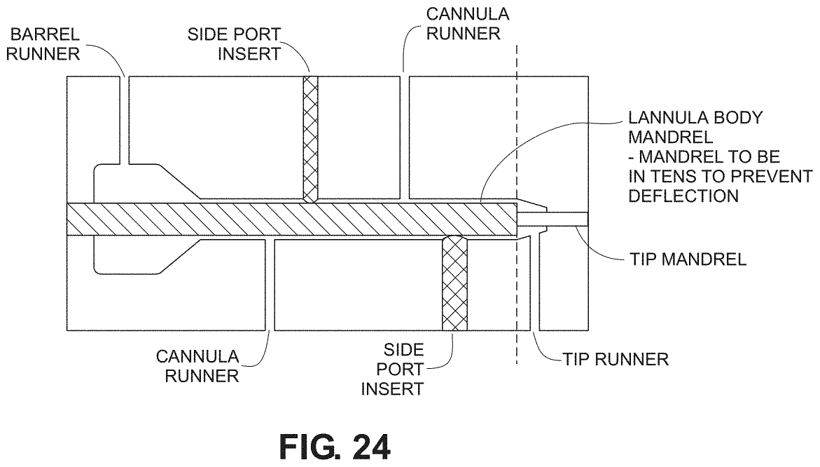

In this and other embodiments, the inserted component or components form the entire assembly when inserted into the mold and injection molded. Because of the inherent nature of injection molding the septum component is not viable in the high-pressure environment required by injection molding and is therefore not part of the finished insert molded component. The septum is installed in a second operation utilizing locating and capture features designed into the component and enabled by the mold, as shown in FIG. 24.

As described above, in order to capture and hold the septum in place the proximal end of the cannula barrel insert molded component has design features that allow a passive capture method possible. Multiple designs are disclosed.

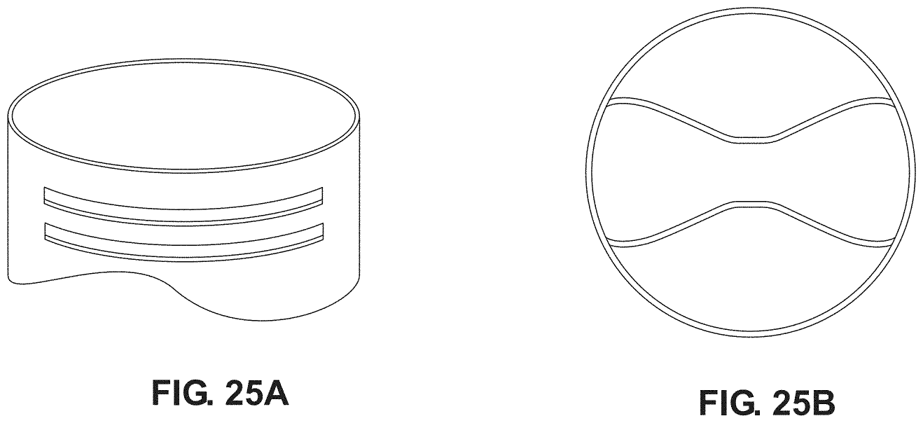

In some embodiments, as shown in FIGS. 25A and 25B, the passive capture features are two parallel slits on mirror sides of the barrel that, when deformed inward toward the longitudinal axis of the component, forms a bar feature that positively captures the septum in-place. FIG. 25B shows a top view of the barrel after bar deformation showing positive septum capture

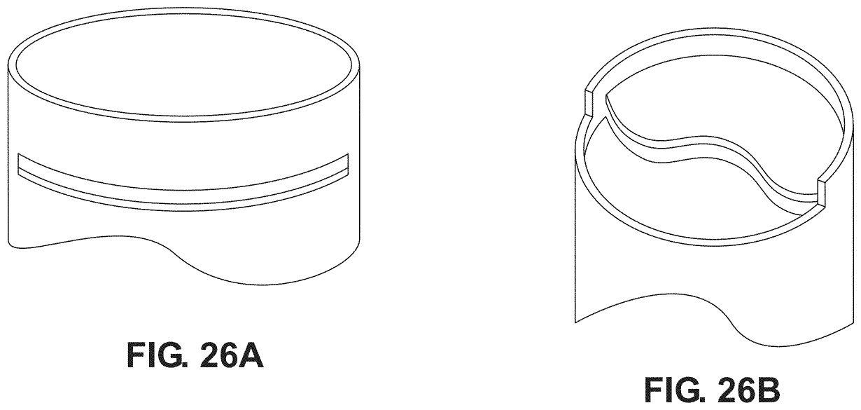

In some embodiments, as shown in FIGS. 26A and 26B, the passive capture feature is one slit on one circumferential side of the barrel that, when deformed inward toward the longitudinal axis of the component, forms a bar feature that positively captures the septum in-place. FIG. 26B shows a top view after bar deformation showing positive septum capture.

It should be understood that any feature described herein with respect to one embodiment can be used in addition to or in place of any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

In embodiments, an insulin delivery system can include a reservoir configured to hold an insulin medication therein, an infusion hub, tubing fluidically connecting the insulin reservoir and the infusion hub and a cannula configured to deliver the insulin medication to a patient. An absorbent element can be positioned within the delivery system and in fluidic contact with the insulin medication, the absorbent element configured to absorb and store preservatives from the insulin medication.

In some embodiments, an impermeable backing layer can be adjacent to the absorbent element and configured to maintain the preservatives within the absorbent.

In some embodiments, the absorbent element comprises EVOH, silicone, a low-density polymer, a PEG block-copolymer (e.g., PETG), PET, nylon, a nylon block-copolymer, a polymeric foam, or a polymeric monolith.

In some embodiments, the absorbent element comprises a preservative capacity greater than a maximum concentration of preservative in the insulin medication.

In some embodiments, the absorbent element is further configured to release preservatives to the insulin medication after storing the preservatives.

In some embodiments, the absorbent element is configured to maintain the preservative concentration at the point of delivery to the patient at a concentration that minimizes local toxicity while maintaining insulin in a stable hexameric state.

In some embodiments, the absorbent element is configured to maintain the preservative concentration at a concentration of greater than about 1.25 mg/mL.

In some embodiments, the absorbent element is configured to maintain the preservative concentration at a concentration of about 1.15-1.75 mg/mL.

In some embodiments, the absorbent element is configured to maintain the preservative concentration at a concentration of about 1.25-1.50 mg/mL.

In some embodiments, the absorbent element comprises an interior layer of the tubing.

In some embodiments, at least a portion of the insulin delivery system includes a barrier layer configured to prevent migration of preservatives from the insulin medication.

In some embodiments, the tubing is a multi-layer tubing, and the barrier layer is at least a portion of a layer of the multi-layer tubing.

In some embodiments, the tubing is a multi-layer tubing, and the barrier layer forms an entire layer of the multi-layer tubing.

In some embodiments, the tubing comprises the barrier layer.

In some embodiments, the barrier layer comprises a coating on the tubing.

In some embodiments, the barrier layer comprises an inner layer of the tubing.

In some embodiments, the barrier layer comprises polyether block-amide, HDPE, polypropylene, PTFE, chloro- and fluorosilicones, hydrochloro-, hydrofluoro-, and perfluoro-polymers, chlorinated polymers (e.g. viton), metal-coated polymers (e.g., mylar), poly carbonate, organic or inorganic plasma-deposited coatings (e.g. PTFE, PVC, halogenated siloxanes, silicon suboxides), vapor-deposited coatings (such as nitrides, titanium nitride, fluorocarbons, metals), Kapton, or parylene.

In some embodiments, a barrel can be connected to the cannula, and the barrier layer is positioned on at least a portion of the barrel.

In some embodiments, a connector can comprise the barrier layer.

In some embodiments, the barrier layer extends through an entire fluid path of the system.

In embodiments, a method of delivering insulin medication can include providing a delivery system, delivering insulin medication comprising preservatives from a reservoir through tubing connecting the reservoir to an infusion hub and delivering the insulin medication to the patient through a cannula connected to the infusion hub, Delivering the insulin medication can include exposing the insulin medication to an absorbent element configured to absorb and store preservatives from the insulin medication.

In some embodiments, the method further comprises the absorbent element can absorb preservatives from the insulin medication.

In some embodiments, the method further comprises the absorbent element releasing preservatives back into the insulin medication.

In some embodiments, the method further comprises preventing preservative evaporation using a barrier layer on one or more components of the delivery system.

In some embodiments, an insulin delivery system can include a reservoir configured to hold an insulin medication therein, an infusion hub, tubing connecting the insulin reservoir and the infusion hub and a cannula configured to deliver the insulin to a patient. A vent in the tubing or the hub can be configured to release preservatives from the insulin medication prior to delivery of the insulin medication to the patient.

In some embodiments, at least a portion of the insulin delivery system includes a barrier layer configured to prevent migration of preservatives from the insulin medication.

In some embodiments, the vent comprises an opening in the barrier layer.

In some embodiments, the vent comprises a portion of the barrier layer that is thinner than other portions of the barrier layer.

In some embodiments, the vent comprises a portion of the barrier layer that is thinner than surrounding portions of the barrier layer.

In some embodiments, the tubing is a multi-layer tubing, and wherein the barrier layer is at least a portion of a layer of the multi-layer tubing.

In some embodiments, the tubing is a multi-layer tubing, and wherein the barrier layer forms an entire layer of the multi-layer tubing.

In some embodiments, the tubing comprises the barrier layer.

In some embodiments, the barrier layer comprises a coating on the tubing.

In some embodiments, the barrier layer comprises an inner layer of the tubing.

In some embodiments, the barrier layer comprises polyether block-amide, HDPE, polypropylene, PTFE, chloro- and fluorosilicones, hydrochloro-, hydrofluoro-, and perfluoro-polymers, chlorinated polymers (e.g. viton), metal-coated polymers (e.g., mylar), poly carbonate, organic or inorganic plasma-deposited coatings (e.g. PTFE, PVC, halogenated siloxanes, silicon suboxides), vapor-deposited coatings (such as nitrides, titanium nitride, fluorocarbons, metals), Kapton, or parylene.

In some embodiments, a barrel can be connected to the cannula, and the barrier layer is positioned on at least a portion of the barrel.

In some embodiments, a connector can comprise the barrier layer.

In some embodiments, the barrier layer extends through an entire fluid path of the system.

In some embodiments, the vent comprises EVOH, silicone, a low-density polymer, a PEG block-copolymer (e.g., PETG), PET, nylon, or a nylon block-copolymer.

In some embodiments, the vent comprises an opening in a wall or layer of the tubing or the hub.

In embodiments, a method of delivering insulin medication can include providing a delivery system, delivering insulin medication comprising preservatives from a reservoir through tubing connecting the reservoir to an infusion hub and delivering the insulin medication to the patient through a cannula connected to the infusion hub. The insulin medication can further be vented, thereby releasing preservatives from the insulin medication.

In some embodiments, the method further comprises preventing preservative loss from the insulin medication by providing a barrier layer along at least a portion of the fluid path of the delivery system.

In some embodiments, venting the insulin medication comprises exposing the insulin medication to an opening in the barrier layer.

In some embodiments, venting the insulin medication comprises exposing the insulin medication to a portion of the barrier layer that is thinner than other portions of the barrier layer.

In some embodiments, venting the insulin medication comprises exposing the insulin medication to a portion the barrier layer that is thinner than surrounding portions of the barrier layer.

In embodiments, an insulin delivery system can include a reservoir configured to hold an insulin medication therein, an infusion hub, tubing connecting the insulin reservoir and the infusion hub and a cannula configured to deliver the insulin medication to a patient. A filter can be configured to capture particulates from the insulin medication prior to delivery of the insulin to the patient.

In some embodiments, the filter comprises features internal to the cannula and in fluidic contact with the insulin configured to affect hydrodynamic flow characteristics of insulin medication flowing within the cannula and to create pressure differential regimes to promote the capture and retention of aggregate particles that have formed out of solution.

In some embodiments, the features repeat along at least a portion of a length of the cannula.

In some embodiments, a ratio of a width of the features and a period of the features is greater than about 1:1 and less than about 1:4.

In some embodiments, the features comprise internally molded features within the cannula.

In some embodiments, the features comprise internally extruded features within the cannula.

In some embodiments, the internal features comprise polyether block-amide.

In some embodiments, the filter comprises an internal coil within the cannula.

In some embodiments, the internal coil comprises a round wire.

In some embodiments, the internal coil comprises a flat wire.

In some embodiments, the internal coil comprises an engineering polymer.

In some embodiments, the internal coil comprises stainless steel.

In some embodiments, the filter comprises a structural component to prevent crushing or kinking of the extruded cannula.

In some embodiments, the filter comprises a threaded inner surface of the cannula.

In some embodiments, the threaded surface comprises pointed threads.

In some embodiments, the threaded surface comprises flat threads.

In some embodiments, the threaded surface comprises angled, overlapping, or buttress style threads.

In some embodiments, at least a portion of the insulin delivery system includes a barrier layer configured to prevent migration of preservatives from the insulin medication.

In some embodiments, the tubing is a multi-layer tubing, and wherein the barrier layer is at least a portion of a layer of the multi-layer tubing.

In some embodiments, the tubing is a multi-layer tubing, and wherein the barrier layer forms an entire layer of the multi-layer tubing.

In some embodiments, the tubing comprises the barrier layer.

In some embodiments, the barrier layer comprises a coating on the tubing.

In some embodiments, barrier layer comprises an inner layer of the tubing.

In some embodiments, the barrier layer comprises polyether block-amide, HDPE, polypropylene, PTFE, chloro- and fluorosilicones, hydrochloro-, hydrofluoro-, and perfluoro-polymers, chlorinated polymers (e.g. viton), metal-coated polymers (e.g., mylar), poly carbonate, organic or inorganic plasma-deposited coatings (e.g. PTFE, PVC, halogenated siloxanes, silicon suboxides), vapor-deposited coatings (such as nitrides, titanium nitride, fluorocarbons, metals), Kapton, or parylene.

In some embodiments, a barrel can be connected to the cannula, and the barrier layer is positioned on at least a portion of the barrel.

In some embodiments, a connector can comprise the barrier layer.

In some embodiments, the barrier layer extends through an entire fluid path of the system.

In some embodiments, a vent in the tubing or the hub can be configured to release preservatives from the insulin medication prior to delivery of the insulin medication to the patient.

In some embodiments, at least a portion of the insulin delivery system includes a barrier layer configured to prevent migration of preservatives from the insulin medication and wherein the vent comprises an opening in the barrier layer.

In some embodiments, at least a portion of the insulin delivery system includes a barrier layer configured to prevent migration of preservatives from the insulin medication and wherein the vent comprises a portion of the barrier layer that is thinner than other portions of the barrier layer.

In some embodiments, at least a portion of the insulin delivery system includes a barrier layer configured to prevent migration of preservatives from the insulin medication and wherein the vent comprises a portion of the barrier layer that is thinner than surrounding portions of the barrier layer.

In some embodiments, an absorbent element can be positioned within the delivery system and in fluidic contact with the insulin medication, the absorbent element configured to absorb and store preservatives from the insulin medication.

In some embodiments, an impermeable backing layer can be adjacent to the absorbent element and configured to maintain the preservatives within the absorbent.

In some embodiments, the absorbent element comprises EVOH, silicone, a low-density polymer, a PEG block-copolymer (e.g., PETG), PET, nylon, a nylon block-copolymer, a polymeric foam, or a polymeric monolith.

In some embodiments, the absorbent element comprises a preservative capacity greater than a maximum concentration of preservative in the insulin medication.

In some embodiments, the absorbent element is further configured to release preservatives to the insulin medication after storing the preservatives.

In some embodiments, the absorbent element is configured to maintain the preservative concentration at the point of delivery to the patient at a concentration that minimizes local toxicity while maintaining insulin in a stable hexameric state.

In some embodiments, the absorbent element comprises an interior layer of the tubing.

In embodiments, a method for delivering insulin can include providing a delivery system, delivering insulin medication comprising preservatives from a reservoir through tubing connecting the reservoir to an infusion hub, filtering the insulin medication to capture particulates from the insulin medication prior to delivery of the insulin medication to the patient and delivering the insulin medication to the patient through a cannula connected to the infusion hub.

In some embodiments, the method further comprises affecting hydrodynamic flow characteristics of insulin medication flowing within the cannula and creating differential pressure regimes to promote the capture and retention of aggregate particles that have formed out of solution.

In some embodiments, filtering the insulin medication comprises providing, within a flow path of the insulin medication, features internal to the cannula, thereby affecting hydrodynamic flow characteristics of insulin medication flowing within the cannula and creating differential pressure regimes to promote the capture and retention of aggregate particles that have formed out of solution.

In some embodiments, filtering the insulin medication comprises using internally molded features within the cannula.

In some embodiments, filtering the insulin medication comprises using internally extruded features within the cannula.

In some embodiments, filtering the insulin medication comprises using a coil internal to the cannula, the coil configured to create a region of features affecting hydrodynamic flow characteristics of insulin medication flowing within the cannula and creating differential pressure regimes to promote the capture and retention of aggregate particles that have formed out of solution.

In some embodiments, the coil comprises a round wire.

In some embodiments, the coil comprises a flat wire.

In some embodiments, the coil comprises stainless steel.

In some embodiments, the coil comprises an engineering polymer.

In some embodiments, the method further comprises exposing the insulin medication to an absorbent element configured to store preservatives from the insulin medication.

In some embodiments, the method further comprises the absorbent element absorbing preservatives from the insulin medication.

In some embodiments, the method further comprises the absorbent element releasing preservatives back into the insulin medication.

In some embodiments, the method further comprises preventing preservative evaporation using a barrier layer on one or more components of the delivery system.

In some embodiments, the method further comprises venting the insulin medication, thereby releasing preservatives from the insulin medication.

In some embodiments, the method further comprises preventing preservative loss from the insulin medication by providing a barrier layer along at least a portion of the fluid path of the delivery system, and wherein venting the insulin medication comprises exposing the insulin medication to an opening in the barrier layer.

In some embodiments, the method further comprises preventing preservative loss from the insulin medication by providing a barrier layer along at least a portion of the fluid path of the delivery system, and wherein venting the insulin medication comprises exposing the insulin medication to a portion of the barrier layer that is thinner than other portions of the barrier layer.

In some embodiments, the method further comprises preventing preservative loss from the insulin medication by providing a barrier layer along at least a portion of the fluid path of the delivery system, and wherein venting the insulin medication comprises exposing the insulin medication to a portion of the barrier layer that is thinner than surrounding portions of the barrier layer.

In embodiments, an insulin delivery system can include a reservoir configured to hold an insulin medication therein, an infusion hub, tubing fluidically connecting the insulin reservoir and the infusion hub; and a cannula configured to deliver the insulin medication to a patient. At least a portion of the insulin delivery system can include a barrier layer configured to prevent migration of preservatives from the insulin medication.

In some embodiments, the tubing is a multi-layer tubing, and wherein the barrier layer is a layer of the multi-layer tubing.

In some embodiments, the barrier layer comprises polyether block-amide, HDPE, polypropylene, PTFE, chloro- and fluorosilicones, hydrochloro-, hydrofluoro-, and perfluoro-polymers, chlorinated polymers (e.g. viton), metal-coated polymers (e.g., mylar), poly carbonate, organic or inorganic plasma-deposited coatings (e.g. PTFE, PVC, halogenated siloxanes, silicon suboxides), vapor-deposited coatings (such as nitrides, titanium nitride, fluorocarbons, metals), Kapton, or parylene.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An insulin delivery system comprising:
a reservoir configured to hold an insulin medication therein;
an infusion hub;
tubing fluidically connecting the insulin reservoir and the infusion hub;
a cannula configured to deliver the insulin medication to a patient; and
an absorbent element positioned within the delivery system and in fluidic contact with the insulin medication, the absorbent element configured to absorb and store preservatives from the insulin medication.

2. The insulin delivery system of claim 1, further comprising an impermeable backing layer adjacent to the absorbent element and configured to maintain the preservatives within the absorbent element.

3. The insulin delivery system of claim 1, wherein the absorbent element comprises EVOH, silicone, a low-density polymer, a PEG block-copolymer (e.g., PETG), PET, nylon, a nylon block-copolymer, a polymeric foam, or a polymeric monolith.

4. The insulin delivery system of claim 1, wherein the absorbent element comprises a preservative capacity greater than a maximum concentration of preservative in the insulin medication.

5. The insulin delivery system of claim 1, wherein the absorbent element is further configured to release preservatives to the insulin medication after storing the preservatives.

6. The insulin delivery system of claim 1, wherein the absorbent element is configured to maintain the preservative concentration at the point of delivery to the patient at a concentration that minimizes local toxicity while maintaining insulin in a stable hexameric state.

7. The insulin delivery system of claim 6, wherein the absorbent element is configured to maintain the preservative concentration at a concentration of greater than about 1.25 mg/mL.

8. The insulin delivery system of claim 6, wherein the absorbent element is configured to maintain the preservative concentration at a concentration of about 1.15-1.75 mg/mL.

9. The insulin delivery system of claim 6, wherein the absorbent element is configured to maintain the preservative concentration at a concentration of about 1.25-1.50 mg/mL.

10. The insulin delivery system of claim 1, wherein the absorbent element comprises an interior layer of the tubing.

11. The insulin delivery system of claim 1, wherein at least a portion of the insulin delivery system includes a barrier layer configured to prevent migration of preservatives from the insulin medication.

12. The insulin delivery system of claim 11, wherein the tubing is a multi-layer tubing, and wherein the barrier layer is at least a portion of a layer of the multi-layer tubing.

13. The insulin delivery system of claim 11, wherein the tubing is a multi-layer tubing, and wherein the barrier layer forms an entire layer of the multi-layer tubing.

14. The insulin delivery system of claim 11, wherein the tubing comprises the barrier layer.

15. The insulin delivery system of claim 11, wherein the barrier layer comprises a coating on the tubing.

16. The insulin delivery system of claim 11, wherein the barrier layer comprises an inner layer of the tubing.

17. The insulin delivery system of claim 11, wherein the barrier layer comprises polyether block-amide, HDPE, polypropylene, PTFE, chloro- and fluorosilicones, hydrochloro-, hydrofluoro-, and perfluoro-polymers, chlorinated polymers (e.g. viton), metal-coated polymers (e.g., mylar), poly carbonate, organic or inorganic plasma-deposited coatings (e.g. PTFE, PVC, halogenated siloxanes, silicon suboxides), vapor-deposited coatings (such as nitrides, titanium nitride, fluorocarbons, metals), Kapton, or parylene.

18. The insulin delivery system of claim 11, further comprising a barrel connected to the cannula, and wherein the barrier layer is positioned on at least a portion of the barrel.

19. The insulin delivery system of claim 11, comprising a connector comprising the barrier layer.

20. The insulin delivery system of claim 11, wherein the barrier layer extends through an entire fluid path of the system.

21-104. (canceled)
